(19) United States
(12) Patent Application Publication
(10) Pub. No.: US 2026/0001905 A1
(43) Pub. Date: Jan. 1, 2026

(54) DIASTEREOMERIC CONJUGATE COMPOSITIONS AND METHODS OF MAKING AND USING SAME

(71) Applicant: Osteoanalgesia, LLC, Cambridge, MA (US)

(72) Inventor: Stephen DiMagno, Chicago, IL (US)

(21) Appl. No.: 19/320,210

(22) Filed: Sep. 5, 2025

Related U.S. Application Data
(63) Continuation of application No. 17/271,847, filed on Feb. 26, 2021.
(60) Provisional application No. 62/725,502, filed on Aug. 31, 2018.

(51) Int. Cl. C07J 41/00; A61K 47/18; A61K 47/58; A61P 19/02; C08L 65/00

(52) U.S. Cl. CPC C07J 41/0005; A61K 47/183; A61K 47/58; A61P 19/02; C08L 65/00; C08L 2205/025

(57) ABSTRACT

Provided are mixtures of dexamethasone-N-methacryloylglycylglycyl hydrazide (MA-Gly-Gly-NHN=Dex) monomers enriched in a desired diastereomer, and polymer compositions of same. Also provided are methods of treating inflammatory conditions such as arthritis that use the polymer compositions.

### RELATED APPLICATIONS

This application claims the benefit of priority to U.S. Provisional Patent Application No. 62/725,502, filed Aug. 31, 2018, the content of which is incorporated herein by reference in its entirety.

### FIELD

This invention relates to biotechnology and more particularly to compositions of defined ratios of water-soluble diastereomers of polymerized forms of a monomer and therapeutic agent, such as MA-Gly-Gly-NHN=Dexamethasone (Dex), for treating arthritis and other inflammatory diseases.

### BACKGROUND

Inflammatory arthritis affects millions of people around the world each year. The treatments currently available are generally limited to reducing disease symptoms and are not curative as such. Rheumatoid arthritis (RA) is the most common inflammatory arthritis and has been reported to affect about one percent of the general population worldwide. In the United States, about 4.5% of people over the age of 55 people have been reported to be affected.

As a symmetric disease, RA usually involves the same joints on both sides of the body. Although its exact cause is unknown, many medications have been developed to relieve its symptoms and slow or halt its progression. Most commonly used medications rest on three principal approaches: symptomatic treatment with non-steroidal anti-inflammatory drugs (NSAIDs), corticosteroid and disease-modifying antirheumatic drugs (DMARDs)

More effective therapies for treating inflammatory conditions such as arthritis are needed.

### SUMMARY

Provided herein are diastereomerically enriched compositions of dexamethasone-N-methacryloylglycylglycyl hydrazide (MA-Gly-Gly-NHN=Dex, or (P-Dex) monomers that can be polymerized to polymers effective in treating inflammatory conditions such as arthritis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF DRAWINGS

FIG. 1 is a graphical representation of the results of process chromatography showing that the two diastereomers of a MMA-dexamethasone monomer are formed in roughly equal amounts (˜0.8:1).

FIG. 2 a graphical representation of the results of process chromatography showing a product that was highly enriched (˜1:60 in the late eluting diastereomer and a filtrate that was enriched in the early eluting diastereomer (˜6:1).

FIG. 3A is graphical representation of the percent area of a F19 NMR for “Bound A” (downfield peak of FIG. 1), “Bound B” (upfield peak of FIG. 1), and “Free dexamethasone.” The stereoisomeric ratio of FIG. 3A is 1:1.5. FIG. 3B is graphical representation of the percent area of a F19 NMR for “Bound A” (downfield peak of FIG. 1), “Bound B” (upfield peak of FIG. 1), and “Free dexamethasone.” The stereoisomeric ratio of FIG. 3A is 1:65.

FIG. 4A is a graphical representation of release rate of Demo 2. Series 4 is the Dex release rate, series 3 is the slow-releasing diastereomer, and series 3 is the fast-releasing diastereomer. FIG. 4B is a graphical representation of release rate of Demo 3. Series 4 is the Dex release rate, series 3 is the slow-releasing diastereomer, and series 3 is the fast-releasing diastereomer.

FIG. 5 is a graphical representation of the effects of the single dose of P-Dex and the 21-day daily dose of P-Dex on collagen-induced arthritis.

FIG. 6 is a table disclosing the results of eight different stability tests over the course of nine months.

### DETAILED DESCRIPTION

Unless defined otherwise, all technical and scientific terms used herein have the same meaning as commonly understood by one of ordinary skill in the art to which the invention belongs. Other more specific definitions are as follows:

The term “alkyl” refers to saturated, straight-or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. In various embodiments, examples of an aryl group may include phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆₋₁₂-aryl). In some embodiments, aryl groups have six carbon atoms (e.g., C₆-aryl).

The terms “alkenyl” or “alkenyl group” mean a branched or straight-chain saturated aliphatic hydrocarbon monovalent radical containing at least one carbon-carbon double bond. This term is exemplified by groups such as ethenyl, propenyl, isopropenyl, and the like.

The terms “ethenyl” and “vinyl” have the same meaning.

The terms “alkynyl” or “alkynyl group” mean a branched or straight-chain saturated aliphatic hydrocarbon monovalent radical containing at least one carbon-carbon triple bond. This term is exemplified by groups such as ethynyl, propynyl, and the like.

The terms “cycloalkyl” or “cycloalkyl group” mean a non-aromatic saturated aliphatic ring of 3-6 carbon atoms. This term is exemplified by groups such as cyclopropyl, cyclobutyl, cyclopentyl, and the like.

The term “acyl” shall be understood to be a C₁-nalkyl with a carbonyl group wherein the point of attachment is via the carbonyl, for example: acetyl (—C(O)CH₃).

The terms “acylating agent” or “acyl transfer reagent” mean a compound that provide an acyl or acetyl group. This term is exemplified by a compound selected from acetyl chloride, acetic anhydride, isopropenyl acetate, vinyl acetate, methyl vinyl acetate, and the like.

The terms “halogen” or “halo” shall be understood to mean bromine, chlorine or iodine.

The phrase “termination product of a radical initiator” shall be understood to mean the radical fragment of any radical initiator. In particular, it shall be understood that the radical termination reaction will occur between the polymer radical and the radical fragment of the radical initiator. For example, the termination product of a radical initiator for azobisisobutyronitrile is

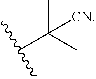 [SMILES: *C(C)(C)C#N]

In another example, the termination product of a radical initiator for 1,1′-azobis(cyclohexanecarbonitrile) is

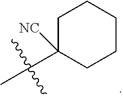 [SMILES: *C1(C#N)CCCCC1]

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

In any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example, all tautomers of a phosphate and a phosphorothioate groups are intended to be included. Furthermore, all tautomers of heterocyclic bases known in the art are intended to be included, including tautomers of natural and non-natural purine-bases and pyrimidine-bases.

Where compounds disclosed herein have unfilled valencies, then the valencies are to be filled with hydrogens or isotopes thereof, e.g., hydrogen-1 (protium) and hydrogen-2 (deuterium).

In embodiments, the compounds described herein are labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In embodiments, the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, the upper and lower limit, and each intervening value between the upper and lower limit of the range is encompassed within the embodiments.

In the event that the structures of compounds disclosed here are in conflict with the nomenclature the compounds are defined by the structure.

The yield of each of the reactions described herein is expressed as a percentage of the theoretical yield.

Compounds can also include their isotopically-labelled forms. Deuterium labelled compounds can be prepared according to known methods using commercially available deuterium labelled reagents (Arisawa, M., et al., Synthesis, 34:138 (2002)).

Optimum reaction conditions, which may include the stoichiometry of reactants and reaction times, may vary depending on the particular reactants used. Unless otherwise specified, reagents, solvents, temperatures, and other reaction conditions may be readily selected by one of ordinary skill in the art.

Amounts and ranges are not meant to be limiting, and increments between the recited percentages and ranges are specifically envisioned as part of the disclosure. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

### Synthesis of Diastereomeric Forms of MA-Gly-Gly-NHN=Dex Monomers and Polymers of Same

The disclosure provides a mixture of diastereomers of dexamethasone-N-methacryloylglycylglycyl hydrazide (MA-Gly-Gly-NHN=Dex) monomers. The diastereomers are presented below as compounds VIa and VIb. In embodiments, the diastereomers are present in the mixture in a diastereomeric ratio (d.r.) of at least 60:40 Formula VIa:Formula VIb, or are present in a d.r. of at least 60:40 of Formula VIb:Formula VIa.

The diastereomer of Formula (VIa) has the structure:

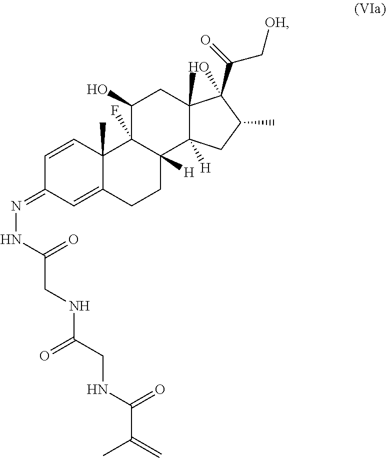 [SMILES: C=C(C)C(=O)NCC(=O)NCC(=O)N/N=C1/C=C[C@@]2(C)C(=C1)CC[C@H]1[C@@H]3C[C@@H](C)[C@](O)(C(=O)CO)[C@@]3(C)C[C@H](O)[C@@]12F]

The diastereomer of Formula (VIb) has the structure:

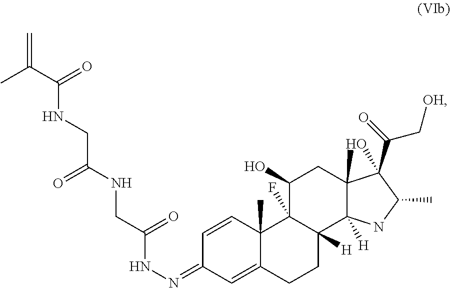 [SMILES: C=C(C)C(=O)NCC(=O)NCC(=O)N/N=C1\C=C[C@@]2(C)C(=C1)CC[C@H]1[C@@H]3N[C@@H](C)[C@](O)(C(=O)CO)[C@@]3(C)C[C@H](O)[C@@]12F]

A mixture comprising a Dex-containing monomer (MA-Gly-Gly-NHN=Dex) enriched for a desired diastereomer can be polymerized using reversible addition-fragmentation transfer (RAFT) polymerization or other living free radical polymerization methods known in the art, e.g., as taught in US 20090311182 (U.S. Ser. No. 12/351,417).

In an embodiment, the CTA RAFT is a dithiobenzoate, trithiocarbonate, or dithiocarbamate RAFT.

In an embodiment, the CTA RAFT is Cyanomethyl methyl(phenyl)carbamodithioate, Cyanomethyl dodecyl trithiocarbonate, 2-Cyano-2-propyl benzodithioate, 4-Cyano-4-(phenylcarbonothioylthio)pentanoic acid, 2-Cyano-2-propyl dodecyl trithiocarbonate, 4-Cyano-4-[(dodecylsulfanylthiocarbonyl)sulfanyl] pentanoic acid, or 2-(dodecylthiocarbonothioylthio)-2-methylpropionic acid.

In an embodiment, the radical initiator is AIBN, TAPB, ACHN, TBHP, BPO, AMBN, ADVN, ACVA, AAPH, or Cumene hydroperoxide.

In an embodiment, the radical initiator is AIBN.

The ratio of compounds VIa and VIb in the mixture can be varied depending on the desired application of the polymer synthesized from the diastereomers. For example, if a more prolonged effect is desired the mixture can be enriched in polymers with the slow-releasing diastereomer. In other embodiments, a more rapid delivery is desired and the mixture can be enriched in the fast-releasing diastereomer.

In addition, conversion of compound VIa to VIb and/or form VIb to VIa can be performed using methods described below.

In embodiments, the monomers comprise an N-(2-hydroxypropyl)methacrylamide, wherein the N-(2-hydroxypropyl)methacrylamide monomer comprises N-(2-hydroxypropyl)methacrylamide and N-methacryloyl glycylglycyl hydrazone glucocorticoid or a pharmaceutically acceptable salt thereof. In embodiments, the glucocorticoid is dexamethasone.

In embodiments, the monomer includes one or more additional moieties, e.g., a (N-methacryloyl glycylglycyl hydrazine) and/or MA-FITC (fluorescein isothiocyanate) via a hydrazone bond as disclosed in, e.g., US20090311182.

If desired, the HPMA copolymer-Dex conjugate enriched in a desired diastereomer can be synthesized with a controllable molecular weight and polydispersity index (PDI). The Dex content can be controlled by the feed-in ratio of MA-Gly-Gly-NHN=Dex. In embodiments, the HPMA copolymer-Dex conjugate has a weight average molecular weight (Mw) of 25-50 kDa, e.g. 30-45 34 kDa and a PDI of, 0.75-2.00, e.g., 0.90-1.75, e.g., 1.10 to 1.75, e.g., 1.20 to 1.50, e.g., 1.34.

If desired, the polymers are copolymers comprising a methacrylamide backbone, wherein the methacrylamide units have alkyl or aryl side chains. In a particular embodiment, the amide group of the methacrylamide backbone is omitted. The polymers may comprise at least one therapeutic agent such as dexamethasone. While the methylacrylamide backbone is described throughout, the polymers of the may also have different backbones, provided they exist in suitable diastereomeric forms, as explained below.

The polymers may optionally further comprise at least one targeting moiety (e.g., linked to the backbone through a linker (e.g., as with the therapeutic agent or imaging agent)).

In an embodiment, the polymers of the complexes are block copolymers. Block copolymers are most simply defined as conjugates of at least two different polymer segments (Tirrel, M. In: Interactions of Surfactants with Polymers and Proteins. Goddard E. D. and Ananthapadmanabhan, K. P. (eds.), CRC Press, Boca Raton, Ann Arbor, London, Tokyo, pp. 59-122, 1992). The simplest block copolymer architecture contains two segments joined at their termini to give an A-B type diblock. Consequent conjugation of more than two segments by their termini yields A-B-A type triblock, A-B-A-B-type multiblock, multisegment A-B-C architectures, and the like. More complex architectures such as (AB)ₙ or AₙBₘ starblocks that have more than two polymer segments linked to a single center, are also encompassed by the instant disclosure.

Also provided are pharmaceutically acceptable salts of diastereomeric forms of mixtures of (VIa) and (VIb) monomers, as well as polymers synthesized with these monomers.

In embodiments, the diastereomers are present in the composition in a diastereomeric ratio (d.r.) of at least 65:35 of Formula VIa:Formula VIb, or are present in a d.r. of at or at least 65:35 of Formula VIb/Formula Via, e.g., the diastereomers are present in the composition in a d.r. of at least 70:30 of Formula VIa:Formula VIb, e.g., 75:25, 80:20, 85:15; 90:10; 95:5; 98:2; 99:1, or 99.9:0.1 of Formula VIa:Formula VIb, or are present in a d.r. of at least 80:20 of Formula VIb/Formula Via, e.g., 75:25, 80:20, 85:15; 90:10; 95:5; 98:2; 99:1, or 99.9:0.1.

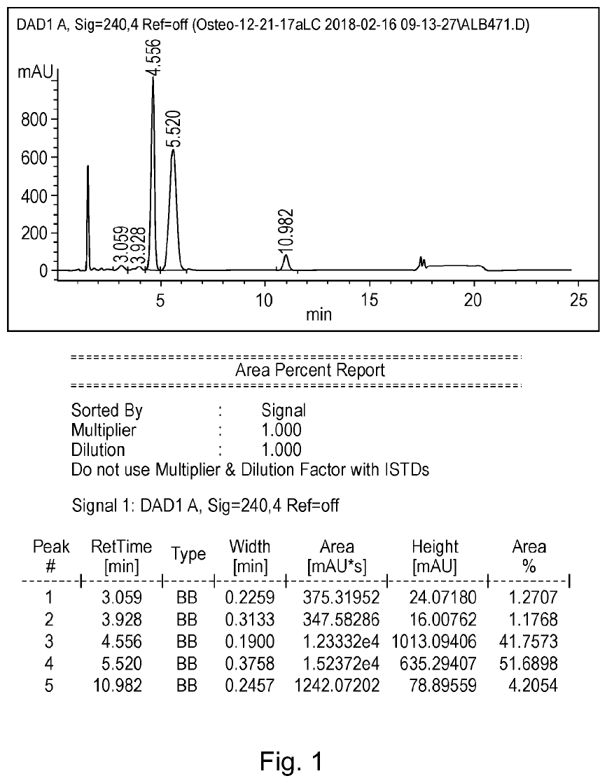

In embodiments, one polymer has a release profile corresponding to the early releasing diastereomer profile shown in FIG. 1.

In embodiments, one polymer has a release profile corresponding to the late releasing diastereomer profile shown in FIG. 1.

The monomer subunits (such as MMA) are typically connected to a therapeutic agent (such as dexamethasone) via a linker. “Linker”, “linker domain”, and “linkage” refer to a chemical moiety comprising a covalent bond or a chain of atoms that covalently attaches at least two compounds, for example, a targeting moiety to a therapeutic agent or connecting an MMA subunit to a therapeutic agent. The linker can be linked to any synthetically feasible position of the therapeutic agent, but preferably in such a manner as to avoid blocking the therapeutic agent's s desired activity. Linkers are generally known in the art. Exemplary linkers may comprise at least one optionally substituted; saturated or unsaturated; linear, branched or cyclic alkyl group or an optionally substituted aryl group. In a particular embodiment, the linker may contain from 0 (i.e., a bond) to about 500 atoms, about 1 to about 100 atoms, or about 1 to about 50 atoms. The linker may also be a polypeptide (e.g., from about 1 to about 20 amino acids). The linker may be biodegradable under physiological environments or conditions. The linker may also be may be non-degradable and can be a covalent bond or any other chemical structure which cannot be cleaved under physiological environments or conditions.

In embodiments, the linker is an alkyl, an aryl, a peptide, or combinations of one or more of an alkyl, aryl or peptide. In embodiments, the linker peptide peptide is, e.g., a gly-gly(n) peptide, where n is 1 to 10.

Diastereomers can be prepared with other glucocorticoids instead of dexamethasone, e.g., monomers can be prepared using hydrocortisone, cortisone, prednisolone, prednisone, methylprednisone, triamcinolone, paramethasone, betamethasone, and fludrocortisone or mixtures thereof.

### Methods of Making HPMA-dexamethasone Monomers Enriched in Diastereomers of Interest

Populations of dexamethasone-containing vinyl monomers enriched in a desired diastereomer can be prepared by
(a) contacting a solution comprising a compound of Formula (IV)

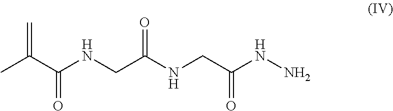

with a compound of Formula (V)

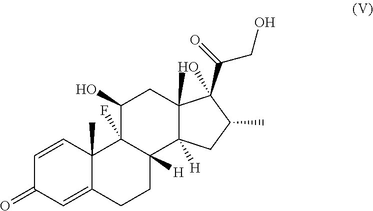 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)CO]

to form a compound of Formula (VI),

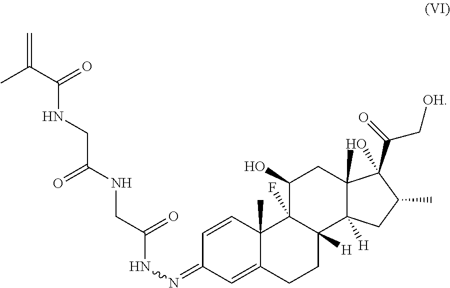 [SMILES: C=C(C)C(=O)NCC(=O)NCC(=O)NN=C1C=C[C@@]2(C)C(=C1)CC[C@H]1[C@@H]3C[C@@H](C)[C@](O)(C(=O)CO)[C@@]3(C)C[C@H](O)[C@@]12F]

The compound of Formula (VI) exists of a mixture of diastereomeric compounds of Formula (VIa) and Formula (VIb):

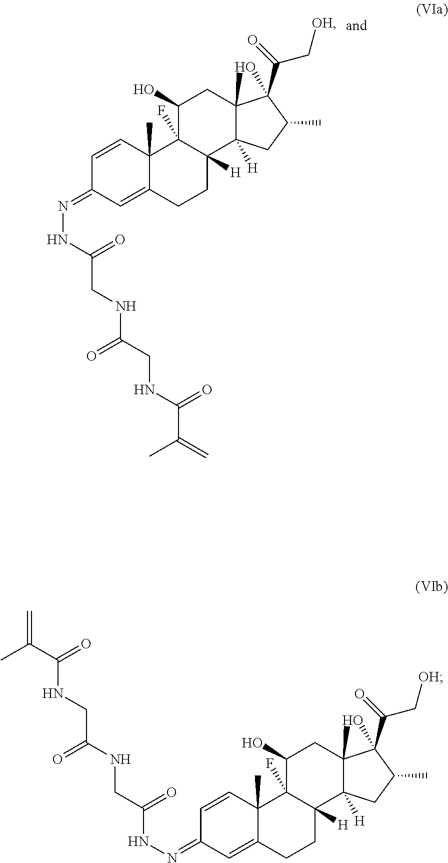

In step (b) solution is removed from the less soluble diastereomer to obtain a solution of VIa and VIb that is diastereomerically enriched with the more soluble diastereomer and a solid that is diastereomerically enriched in the less soluble diastereomer.

Optionally, the method additionally comprises equilibrating the non-desired diastereomer, and repeating steps (a) and (b).

The mixture of Formula (VIa) and (VIb) is then contacted with a compound of Formula (VII)

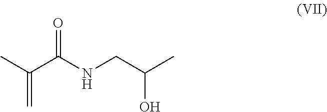

to form an HPMA-dexamethasone monomer conjugate enriched in one diastereomer of the HPMA-dexamethasone monomer conjugate.

A monomer of Formula VIa or Formula VIb can alternatively be prepared by a method comprising contacting in a step (a) a solution comprising a compound of Formula (IV):

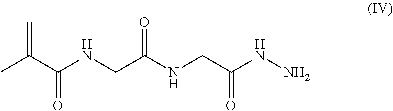

with a compound of Formula (V):

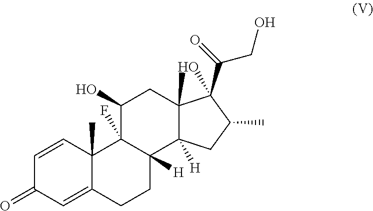 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)CO]

to form a compound of Formula (VI),

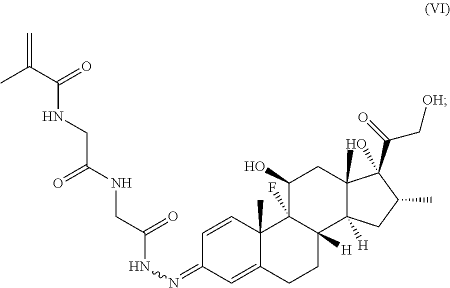 [SMILES: C=C(C)C(=O)NCC(=O)NCC(=O)NN=C1C=C[C@@]2(C)C(=C1)CC[C@H]1[C@@H]3C[C@@H](C)[C@](O)(C(=O)CO)[C@@]3(C)C[C@H](O)[C@@]12F]

The compound of Formula (VI) comprises diastereomeric compounds of Formula (VIa) and Formula (VIb):

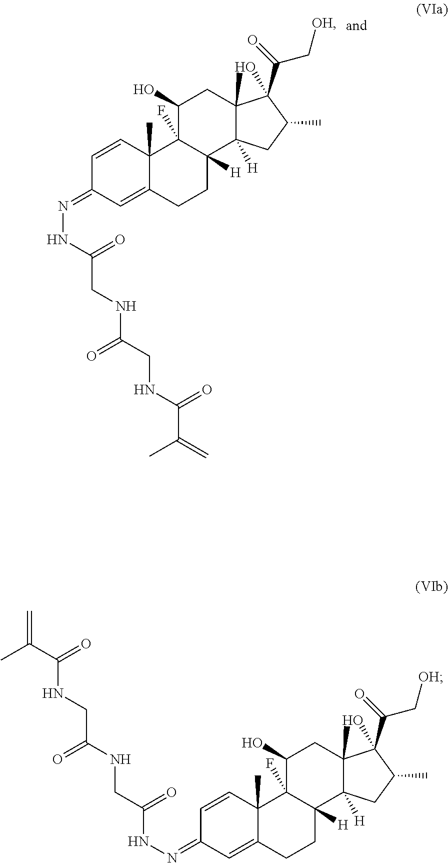

In a second step (b) the less soluble diastereomer is removed from the solution to obtain a solution of VIa and VIb that is diastereomerically enriched in the more soluble diastereomer and a solid that is diastereomerically enriched in the less soluble diastereomer.

Optionally, the method includes in a step (c) racemizing the less-soluble diastereomer, and repeating steps (a) and (b).

In embodiments, the solvent of step (a) is a polar protic solvent.

In embodiments, the solvent of step (a) is methanol.

In embodiments, the solvent of step (a) includes a catalyst.

In embodiments, the catalyst is an acid catalyst, e.g., hydrochloric acid.

In embodiments, the less-soluble diastereomer of step (b) is removed from the solution as a solid.

In embodiments, the less-soluble diastereomer of step (b) is recrystallized in THF and MTBE. In embodiments, the solid obtained in step (b) is a ratio of 60:1 less-soluble to more-soluble diastereomer.

### Pharmaceutical Compositions of HPMA-dexamethasone Polymers Enriched in a Desired Diastereomer

The disclosure additionally provides pharmaceutical composition comprising at least copolymer based on the diastereomerically enriched monomers described herein, along with at least one pharmaceutically acceptable carrier.

In an embodiment, the composition comprises a mixture of HPMA-dexamethasone conjugate copolymers, wherein the composition comprises interspersed MMA-dexamethasone monomers, e.g., subunits of B and C of Formula (III) or a pharmaceutically acceptable salt thereof.

In embodiments, the composition of Formula (III) comprising a mixture of HPMA-dexamethasone conjugate copolymers, wherein the composition comprises interspersed diastereomers B and C or a pharmaceutically acceptable salt thereof,
wherein the polymer of Formula (III) is:

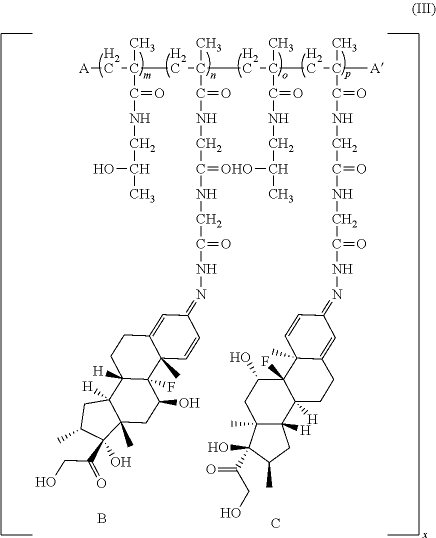

wherein:
m is an integer between 0 or 1 and 1000;
n is an integer between 0 or 1 and 1000;
o is an integer between 0 or 1 and 1000;
p is an integer between 0 or 1 and 1000;
X is an integer between 0 or 1 and 1000;
   wherein A and A′ are each, independently, selected from H or a termination product of a radical initiator, provided that at least one A or A′ is H; and
wherein the diastereomers comprising each of the copolymers are present in the mixture in a diastereomeric ratio (d.r.) of at least 60:40 B:C, or are present in a d.r. of at or at least 60:40 of C:B.

In an embodiment, the copolymer includes m, n, o, and p moieties in any sequence or order.

In an embodiment, A and A′ are each, independently, selected from H or

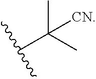 [SMILES: *C(C)(C)C#N]

provided that at least one A or A′ is H; and
wherein the copolymers are enriched for one diastereomer of a HPMA-dexamethasone conjugate.

In an embodiment, the end groups are any suitable structure or structures that protects the copolymers from degradation.

In an embodiment, the end group or groups is a termination product of a radical initiator is from AIBN, TAPB, ACHN, TBHP, BPO, AMBN, ADVN, ACVA, AAPH, or Cumene hydroperoxide.

In an embodiment, termination product of a radical initiator is

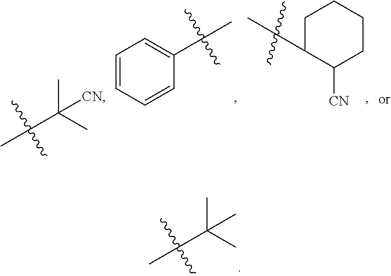

In an embodiment, A′ is selected from H or

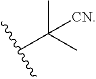 [SMILES: *C(C)(C)C#N]

In an embodiment, A is selected from the terminal fragment of the CTA RAFT.

In an embodiment, A is

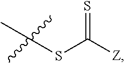

—SH, or has been eliminated to make a double bond, and wherein Z is selected from the group consisting of —S(C₁-C₃₀alkyl), —S(C-C₃₀aryl), C₁-C₃₀alkyl, or C₆-C₃₀aryl.

In an embodiment, the copolymers are present in the mixture in a diastereomeric ratio (d.r.) of at least 60:40 B:C, or are present in a d.r. of at or at least 60:40 of C:B, or a pharmaceutically acceptable salt thereof.

In another embodiment, the diastereomers are present in the composition in a d.r. of at least 65:35 of B:C, or are present in a d.r. of at or at least 65:35 of C:B.

In a further embodiment, the diastereomers are present in the composition in a d.r. of at least 70:30 of B:C, or are present in a d.r. of at least 70:30 of C:B.

In yet another embodiment, the diastereomers are present in the composition in a d.r. of at least 75:25 of B:C, or are present in a d.r. of at least 75:25 of C:B.

In an embodiment, the diastereomers are present in the composition in a d.r. of at least 80:20 of B:C, or are present in a d.r. of at least 80:20 of C:B.

In a further embodiment, the diastereomers are present in the composition in a d.r. of at least 85:15 of B:C, or are present in a d.r. of at least 85:15 of C:B.

In another embodiment, the diastereomers are present in the composition in a d.r. of at least 90:10 B:C, or are present in a d.r. of at least 90:10 of C:B.

In an embodiment, the diastereomers are present in the composition in a d.r. of at least 95:5 of B:C, or are present in a d.r. of at least 95:5 of C:B.

In another embodiment, the diastereomers are present in the composition in a d.r. of at least 98:2 of B:C, or are present in a d.r. of at least 98:2 of C:B.

In a further embodiment, the diastereomers are present in the composition in a d.r. of at least 99:1 of B:C, or are present in a d.r. of at least 99:1 of C:B.

In an embodiment, the diastereomers are present in the composition in a d.r. of at least 99.9:0.1 of B:C, or are present in a d.r. of at least 99.9:0.1 of C:B.

In an embodiment, the average molar mass of the HPMA-dexamethasone conjugate copolymers is between 11,000-49,000 g/mol.

In another embodiment, the average molar mass of the HPMA-dexamethasone conjugate copolymers is between 20,000-40,000 g/mol.

In yet another embodiment, the average molar mass of the HPMA-dexamethasone conjugate copolymers is between 25,000-35,000 g/mol.

In an embodiment, the polydispersity index of the HPMA-dexamethasone conjugate copolymers is between 1.16-1.63.

In an embodiment, the polydispersity index of the HPMA-dexamethasone conjugate copolymers is between 1.25-1.50.

In an embodiment, the percent weight of dexamethasone in the HPMA-dexamethasone conjugate copolymers is between 9-12%.

In an embodiment, the percent weight of dexamethasone in the HPMA-dexamethasone conjugate copolymers is between 10-11%.

The composition may further include at least one other therapeutic agent, e.g., an anti-inflammatory therapeutic agent. Such composition may be administered, in a therapeutically effective amount, to a patient in need thereof for the treatment and/or imaging of an inflammatory disease or disorder. In a particular embodiment, at least one other anti-inflammatory agent is administered separately from the above composition (e.g., sequentially or concurrently).

The compositions disclosed herein can be administered by any suitable route, for example, by injection (e.g., for local (direct) or systemic administration (intravenous)), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intraocular, intrapulmonary, intraarterial, intrarectal, intramuscular, and intranasal administration. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition disclosed herein. See, e.g., Remington's Pharmaceutical Sciences, 18th Ed. (1990, Mack Publishing Co., Easton, Pa. 18042) pages 1435 1712 which are herein incorporated by reference. The pharmaceutical composition can be prepared, for example, in liquid form, or can be in dried powder form (e.g., lyophilized).

In a particular embodiment, the composition may be administered topically. The composition for topical administration may be formulated, for example, as a cream, lotion, foam, or ointment. As another example, inflammations of the joints or tendons (e.g., arthritis, tendonitis) may be treated by injecting the composition directly into the affected location. Such injections may be administered at intervals until inflammation has subsided. As another example, inflammatory conditions of the airways or lungs (e.g., asthma) may be treated by inhalation therapy with an aerosol formulation of the composition. As still another example, inflammations or autoimmune diseases of the gastrointestinal tract (e.g., irritable bowel syndrome, Crohn's Disease) may be treated orally with the composition formulated as a pill, powder, capsule, tablet, or liquid to coat the lumenal surface of the gastrointestinal tract. As another example, the composition may be administered systemically (e.g., intravenously) for treatment of inflammatory disorders that are, at least in part, systemic in nature (e.g., systemic lupus, multiple sclerosis, rheumatoid arthritis, dermatomyositis and scleroderma) or that do not lend themselves well to localized drug delivery.

If desired, the pharmaceutical compositions provided herein can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115 138). In particular, a controlled release device can be introduced into an animal in proximity to the site of inappropriate inflammation. Other controlled release systems are discussed in the review by Langer (Science (1990) 249:1527 1533).

A composition disclosed herein may be administered for immediate relief of acute symptoms or may be administered regularly over a time course to treat and/or image the inflammatory disorder. The dosage ranges for the administration of the composition are those large enough to produce the desired effect (e.g., curing, relieving, and/or preventing the inflammatory disorder, the symptom of it, or the predisposition towards it). The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

### Methods of Treatment using HPMA-dexamethasone Polymers Enriched in a Desired Diastereomer

As used herein, “treatment” is an approach for obtaining beneficial or desired clinical results.

An “individual” or a “subject” is a mammal, more preferably, a human. Mammals also include, but are not limited to, farm animals (e.g., cows, pigs, horses, chickens, etc.), sport animals, pets, primates, horses, dogs, cats, mice and rats.

In an embodiment, the pharmaceutical composition includes a diastereomerically-enriched polymer based on an HPMA monomer linked to dexamethasone. In embodiments, the diastereomically-enriched composition will include additional inflammation resolution drugs, anti-inflammatory drugs, anti-arthritic drugs, targeting moieties, and imaging agents, as used herein, include acceptable salts, esters, or salts of such esters. For example, glucocorticoids include pharmaceutically acceptable salts and esters thereof, therefore, when a drug is described, e.g., dexamethasone, pharmaceutically acceptable salts thereof are also described, such as dexamethasone palmitate.

The term “pharmaceutically acceptable salts” refers to physiologically and pharmaceutically acceptable salts of the compounds disclosed herein: i.e., salts that retain the desired biological activity of the parent compound and do not impart undesired toxicological effects thereto.

Pharmaceutically acceptable base addition salts and acid addition salts are known in the art (see, for example, Berge et al., “Pharmaceutical Salts,” J of Pharma Sci., 1977, 66:1-19; REMINGTON'S PHARMACEUTICAL SCIENCES, 18th Ed. (1990, Mack Publishing Co., Easton, PA); and GOODMAN AND GILMAN'S, THE PHARMACOLOGICAL BASIS OF THERAPEUTICS (10th ed. 2001)).

The term “prodrug” indicates a therapeutic agent that is prepared in an inactive form that is converted to an active form (i.e., drug) within the body or cells thereof by the action of endogenous enzymes or other chemicals and/or conditions.

In addition, currently available protein drugs and orally available low molecular weight drug may also benefit from the principles illustrated herein. For example, the extravasation of the injected polymer into the RA joints was delayed for 1 to 2 hours. Thus, for the protein or peptide drugs, they must survive this period of time against hepatic and renal clearance. Protein or peptide drugs may be stabilized by methods known in the art, for example, PEGylation of the protein and/or modification of the polymer backbone may provide a beneficial means in solving this problem (Smolen and Steiner, Therapeutic strategies for rheumatoid arthritis. Nature Review Drug Discovery (2003) 2:473-488).

The compositions may optionally include one or more targeting moieties, which may be used to direct a delivery system to a specific tissue, such as bone, cartilage, or certain cell types, etc. Illustrative examples of targeting moieties include, but are not limited to, folic acid, mannose, bisphosphonates, quaternary ammonium groups, peptides (e.g., oligo-Asp or oligo-Glu), aminosalicylic acid, and/or antibodies or fragments or derivatives thereof (e.g., Fab, humanized antibodies, and/or single chain variable fragment (scFv)). A targeting moiety may be linked to the polymer backbone via covalent or physical bonds (linkages).

The bio-assay label, therapeutic agent, and/or targeting moiety may be linked to the water-soluble polymer backbone by way of a spacer. Spacers are known in the art and the person of ordinary skill in the art may select a spacer based on length, reactivity, flexibility and the like. For example, a spacer may be an alkyl or alkyne having from one to 50, preferably one to 15 carbons.

A spacer may be a peptide sequence (for example, selected from all nature amino acids) having from one to 20, preferably one to 10 residues. In yet another example, a spacer may contain a hydrazone bond which is cleavable under acidic pH. These spacers may be cleaved upon a stimulus including, but not limited to, changes in pH, presence of a specific enzyme activity (for example, cathepsins (e.g., cathepsin K), MMPs, etc.), changes in oxygen levels, etc. Optionally, the spacers between a targeting moiety and the polymer backbone may be cleaved upon a stimulus including, but not limited to, changes in pH, presence of a specific enzyme activity (for example, cathepsins (e.g., cathepsin K), MMPs, etc.), changes in oxygen levels, etc.

Optionally, the spacers between the therapeutic agent and the polymer backbone may be cleaved upon a stimulus including, but not limited to, changes in pH, presence of a specific enzyme activity (for example, cathepsins (e.g., cathepsin K), MMPs, etc.), changes in oxygen levels, etc.

Optionally, a bio-assay label/imaging agent (or labels) may be attached to the polymer backbone. It may be any label known in the art, including, but not limited to, an optical imaging agent, fluorescent probe, MRI contrast agent, radioisotope, biotin, gold, etc. Their average mol percentage per polymer chain may range from 0% to about 50%.

Optionally, the polymer is cross-linked, e.g., have a biodegradable cross-linkage, to a certain degree, the linear polymer backbone. The resulting delivery system still retains its water-solubility. The linkage itself is preferably cleavable under physiological conditions.

As will be appreciated by a person of ordinary skill in the art, each class (e.g., therapeutic agent, targeting moiety, bio-assays label and/or imaging agent, spacer) may comprise any number of different compounds or compositions. For example, the therapeutic agent may consist of a mixture of one or more NSAIDs and one or more glucocorticoid, such as a combination of dexamethasone and hydrocortisone. Therefore, the compounds and methods disclosed herein advantageously allow for any combination of different therapeutic agents, targeting moieties, bio-assays labels, spacers and/or imaging agents to be incorporated onto the water-soluble polymer backbone. As a result, a drug delivery or imaging system can be created with two or more different therapeutic agents and/or two or more different targeting moieties and/or two or more different bio-assays labels, and/or two or more different spacers (one or more of which may be cleavable, wherein the cleavage stimulus may be different for different spacers) and/or two or more imaging agents. For example, one or more imaging agents may be combined with one or more therapeutic agents, to produce a drug/imaging agent combination, which, for example, may be used to treat and/or monitor the subject. One exemplary embodiment of such a drug/imaging agent is a method of determining the effects of a particular drug or drug combination. For example, the drug/imaging agent may contain a candidate drug wherein the imaging agent allows for enhanced monitoring of the candidate drugs effects. In another exemplary embodiment, the drug/imaging agent may also be used to treat a subject and to monitor the subject's response to the treatment.

An effective amount of a drug is well known in the art and changes due to the age, weight, severity of a subject's condition, the particular compound in use, the strength of the preparation, and the mode of administration. The determination of an effective amount is preferably left to the prudence of a treating physician, but may be determined using methods well known in the art (The Pharmacological Basis of Therapeutics, 10^{th} ed, Gilman et al. eds., McGraw-Hill Press (2001); Remington's Pharmaceutical Science's, 18th ed. Easton: Mack Publishing Co. (1990)). The compositions disclosed herein may be prepared using methods known in the art, for example, the preparation of a pharmaceutical composition is known in the art (The Pharmacological Basis of Therapeutics, 10^{th} ed., Gilman et al. eds., McGraw-Hill Press (2001); Remington's Pharmaceutical Science's, 18th ed. Easton: Mack Publishing Co. (1990)).

The compositions may be administered by any desirable and appropriate means. For in vivo delivery (i.e., to a subject having arthritis or other inflammatory diseases), it is preferred that the delivery system be biocompatible and preferably biodegradable and non-immunogenic. In addition, it is desirable to deliver a therapeutically effective amount of a compound in a physiologically acceptable carrier. Injection into an individual may occur subcutaneous, intravenously, intramuscularly, intraperitoneal, intraarticular or, for example, directly into a localized area. Alternatively, in vivo delivery may be accomplished by use of a syrup, an elixir, a liquid, a tablet, a pill, a time-release capsule, an aerosol, a transdermal patch, an injection, a drip, an ointment, etc.

As stated hereinabove, the polymers disclosed herein are typically used to treat and/or monitor at least one inflammatory disease/disorder. As used herein, the terms “inflammatory disease” and “inflammatory disorder” refer to a disease or disorder caused by or resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death. An “inflammatory disease” can be either an acute or chronic inflammatory condition and can result from infections or non-infectious causes. Inflammatory diseases and disorders include, without limitation, inflammatory lesions (e.g., those associated with multiple sclerosis, graft or organ transplant rejection, tuberculosis, and the like), atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, eczema, dermatitis, cystic fibrosis, temporomandibular joint syndrome (TMJ), chronic obstructive pulmonary disease, inflamed nerve root, arthrosteitis, rheumatoid arthritis, osteoarthritis (OA), inflammatory arthritis, Sjogren's Syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroditis, Graves' disease, Goodpasture's disease, mixed connective tissue disease, sclerosing cholangitis, inflammatory bowel disease, colitis, Crohn's Disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, emphysema, hay fever, allergies, acute anaphylaxis, rheumatic fever, glomerulonephritis, pyelonephritis, cellulitis, cystitis, chronic cholecystitis, ischemia (ischemic injury and ischemia-reperfusion), hypertension, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, and vulvovaginitis, angitis, chronic bronchitis, osteomylitis, optic neuritis, temporal arteritis, transverse myelitis, necrotizing fascilitis, necrotizing enterocolitis, infection-related disorders such as acute and chronic bacterial and viral infections and sepsis, and neoplasia (including leukocyte recruitment in cancer and angiogenesis). In a particular embodiment, the inflammatory disease is selected from the group consisting of rheumatoid arthritis, osteoarthritis (OA), temporomandibular joint syndrome (TMJ), inflamed nerve root, Crohn's disease, chronic obstructive pulmonary disease, psoriasis diseases, asthma, colitis, multiple sclerosis, lupus, erythematosus, and atherosclerosis. In one embodiment, the inflammatory disease is rheumatoid arthritis.

In a further embodiment, provided herein is a method of treating an inflammatory disease in a subject in need thereof comprising administering to the subject a therapeutically effective amount the mixture of HPMA-dexamethasone conjugate copolymers of Formula (III) or a pharmaceutical composition thereof.

The linkages (linker domains) of the instant polymers may be non-degradable or degradable under physiological conditions. The linkers may be cleavable by a protease such as cathepsins and MMPS. The linkers may also be pH sensitive (e.g., cleaved under acidic conditions (e.g., pH<6, preferably <5.5). In a particular embodiment, the pH sensitive linker comprises a hydrazone bond, acetal bond, cis-aconityl spacer, phosphamide bond, silyl ether bond, and the like.

The instant disclosure also encompasses compositions comprising at least copolymer based on the diastereomerically enriched monomers disclosed herein and at least one pharmaceutically acceptable carrier. The composition may further comprise at least one other anti-inflammatory therapeutic agent. Such composition may be administered, in a therapeutically effective amount, to a patient in need thereof for the treatment and/or imaging of an inflammatory disease or disorder. In a particular embodiment, at least one other anti-inflammatory agent is administered separately from the above composition (e.g., sequentially or concurrently).

The compositions disclosed herein can be administered by any suitable route, for example, by injection (e.g., for local (direct) or systemic administration (intravenous)), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intraocular, intrapulmonary, intraarterial, intrarectal, intramuscular, and intranasal administration. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical disclosed herein. See, e.g., Remington's Pharmaceutical Sciences, 18th Ed. (1990, Mack Publishing Co., Easton, Pa. 18042) pages 1435-1712 which are herein incorporated by reference. The pharmaceutical compositions can be prepared, for example, in liquid form, or can be in dried powder form (e.g., lyophilized).

In a particular embodiment, the composition is administered topically. The composition for topical administration may be formulated, for example, as a cream, lotion, foam, or ointment. As another example, inflammations of the joints or tendons (e.g., arthritis, tendonitis) may be treated by injecting the composition directly into the affected location. Such injections may be administered at intervals until inflammation has subsided. As yet another example, inflammatory conditions of the airways or lungs (e.g., asthma) may be treated by inhalation therapy with an aerosol formulation of the composition. As still another example, inflammations or autoimmune diseases of the gastrointestinal tract (e.g., irritable bowel syndrome, Crohn's Disease) may be treated orally with the composition formulated as a pill, powder, capsule, tablet, or liquid to coat the lumenal surface of the gastrointestinal tract. As another example, the composition may be administered systemically (e.g., intravenously) for treatment of inflammatory disorders that are, at least in part, systemic in nature (e.g., systemic lupus, multiple sclerosis, rheumatoid arthritis, dermatomyositis and scleroderma) or that do not lend themselves well to localized drug delivery.

In yet another embodiment, the pharmaceutical compositions disclosed herein can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190;During et al., Ann. Neurol. (1989) 25:351; Howard et al., J. Neurosurg. (1989) 71:105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115 138). In particular, a controlled release device can be introduced into an animal in proximity to the site of inappropriate inflammation. Other controlled release systems are discussed in the review by Langer (Science (1990) 249:1527 1533).

The compositions may be administered for immediate relief of acute symptoms or may be administered regularly over a time course to treat and/or image the inflammatory disorder. The dosage ranges for the administration of the composition are those large enough to produce the desired effect (e.g., curing, relieving, and/or preventing the inflammatory disorder, the symptom of it, or the predisposition towards it). The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The invention will be further illustrated in the following non-limiting examples.

### EXAMPLES

### Example 1. Synthetic Procedures for Forming Dexamethasone-MMA Conjugate (MA-Gly-Gly-NHN=Dex) (“P-Dex-monomer”) Compounds

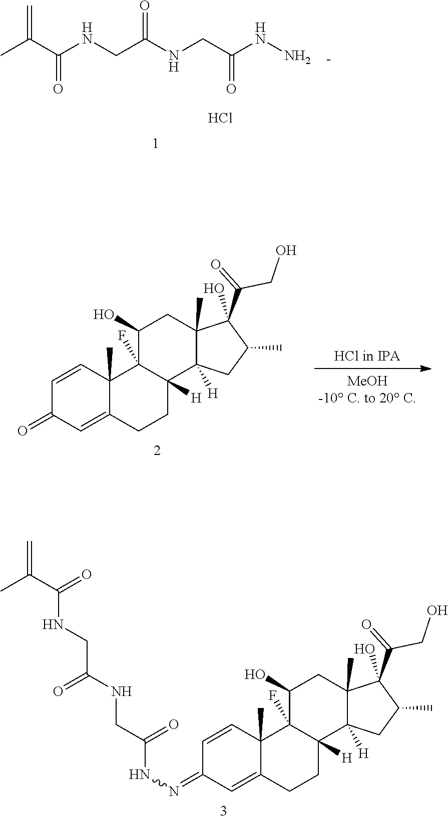 [SMILES: C=C(C)C(=O)NCC(=O)NCC(=O)NN ; Cl ; C[C@@H]1C[C@H]2[C@@H]3CCC4=CC(=O)C=C[C@]4(C)[C@@]3(F)[C@@H](O)C[C@]2(C)[C@@]1(O)C(=O)CO ; C=C(C)C(=O)NCC(=O)NCC(=O)NN=C1C=C[C@@]2(C)C(=C1)CC[C@H]1[C@@H]3C[C@@H](C)[C@](O)(C(=O)CO)[C@@]3(C)C[C@H](O)[C@@]12F]

P-Dex monomer compounds were prepared using the following procedure. A 10 g (dexamethasone, 1 equiv, 10 g; compound 1, 1.3 equiv, 8.3 g; 5 N HCl, 2.5 mL; methanol, 200 mL) demonstration reaction was performed at −10° C. and in-process chromatography (IPC) after 18 hours confirmed only 11.1% dexamethasone remained. The reaction was quenched with 10% aqueous potassium acetate, warmed to room temperature, and stirred for 18 hours. The batch became a white suspension and product was isolated by filtration. The solid cake was washed with THF:MTBE (˜1:1), MTBE and conditioned for 3 hours to afford 6.9 g off-white solid (46% yield) with 98.7 area % purity determined by HPLC. The reaction was scaled to 100 g to provide additional Dex monomer. The reaction in-process chromatography at 18 hours confirmed only 4.2% dexamethasone remained. The batch was quenched and isolated in the same manner as the 10 g experiment and the isolated solid was dried under vacuum at 20-30° C. to afford 86.2 g Dex monomer as an off-white solid (58% yield) with 99.2% purity (with an isomeric ratio of 1.5%:97.7%).

### Example 2. Isolation of a Less-Soluble P-Dex Monomer Diastereomer

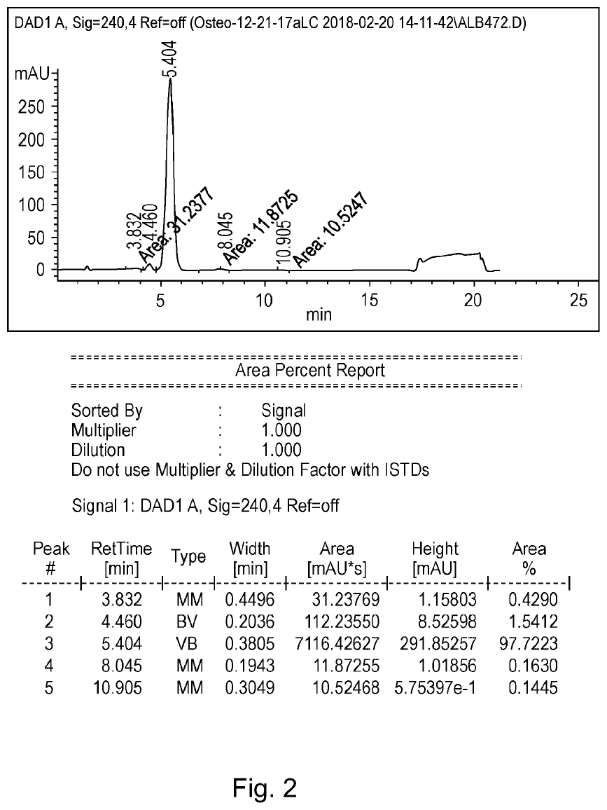

In-process chromatography of the condensation reaction indicated that the two diastereomers of the monomer product were formed in roughly equal amounts (˜0.8:1, see FIG. 1). Isolation by precipitation in this manner gave a product that was highly enriched (˜1:60, FIG. 2) in the late eluting diastereomer and a filtrate that was enriched in the early eluting diastereomer (˜6:1). These result, taken together, indicate that the late eluting diastereomer is much less soluble than the early eluting diastereomer in MTBE, and that MTBE precipitation provides and easy method to separate the diastereomers.

### Example 3 Interconversion of the P-Dex Monomer Diastereomers

The equilibration of P-Dex monomer diastereomers is performed in methanol by heating a purified P-Dex monomer (isomeric ratio 1:60) in 8 or more volumes of methanol at 65° C. for 24 h, which is a sufficient period of time to equilibrate the mixture to a 1.1:1 ratio of diastereomers.

### Example 4 Polymerization of P-Dex Monomer Enriched in a Single Diastereomer

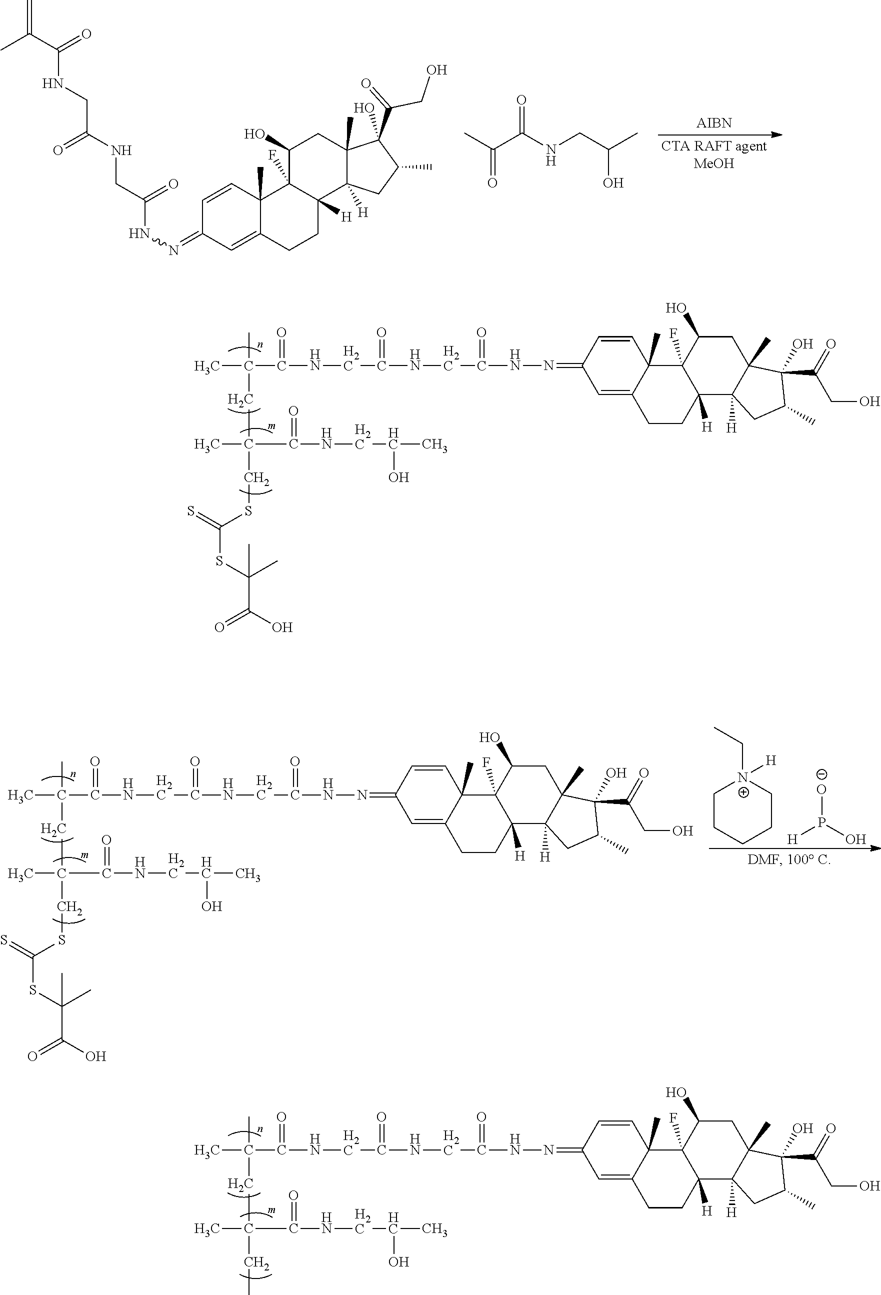

Using AIBN (170 mg, 1.04 mmol, 0.04 equiv) as the initiator and S,S′-bis(α,α′-dimethyl-α″-acetic acid)-trithiocarbonate as a RAFT agent (637 mg, 2.25 mmol, 0.09 equiv), the less-soluble monomer of Example 2 (14.3 g, 24.3 mmol, 1 equiv), which was completely dissolved in degassed methanol, was copolymerized with HPMA (69.1 g, 483 mmol, 19.8 equiv). After purification with LH-20 column and dialysis, the GPC analysis results showed the Mₙ to be 48,875 g/mol with a polydispersity index of 1.56, and 11% dexamethasone by weight. (isomeric ratio 1:65).

The protected polymer (28.5 g, 0.95 mmol, 1 equiv) was deprotected using 1-ethylpiperidin-1-ium hydrogen phosphonate in DMF at 100° C. to yield P-Dex.

### Example 5 Monomer Release from Diastereomerically-enriched P-Dex Polymers as Measured by Quantitative 19F-NMR

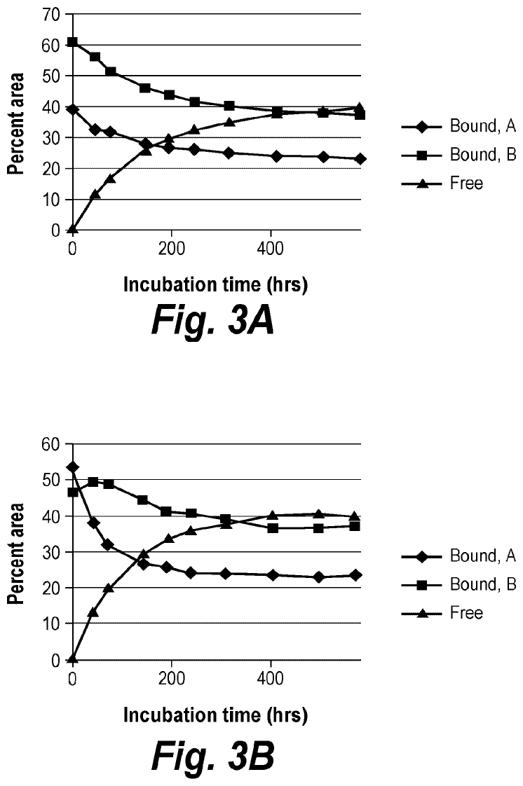

Quantitative 19F-NMR experiments were performed on two different lots of P-Dex polymers contained (as determined by 19F NMR) two different ratios of diastereomeric hydrazone linkages. Concentrated solutions of these polymers were prepared in pH=4.0 buffer, and the release of free Dex was monitored over the course of three weeks. Kinetic modeling of the data from the Dex release experiments indicates that the release rate constants from the two diastereomers differ by a factor of three, thus, it is possible to prepare polymers featuring more rapid release or less rapid release by changing the diastereomeric ratio of the polymer-to-Dex hydrazone linkages (see FIGS. 3A and 3B).

### Example 6. An Empirical Kinetic Model of Dex Release from P-Dex Polymers

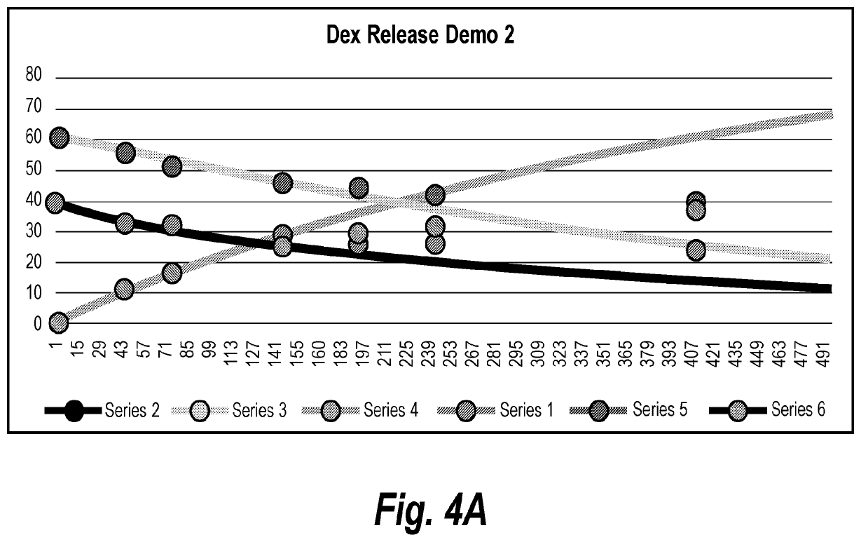

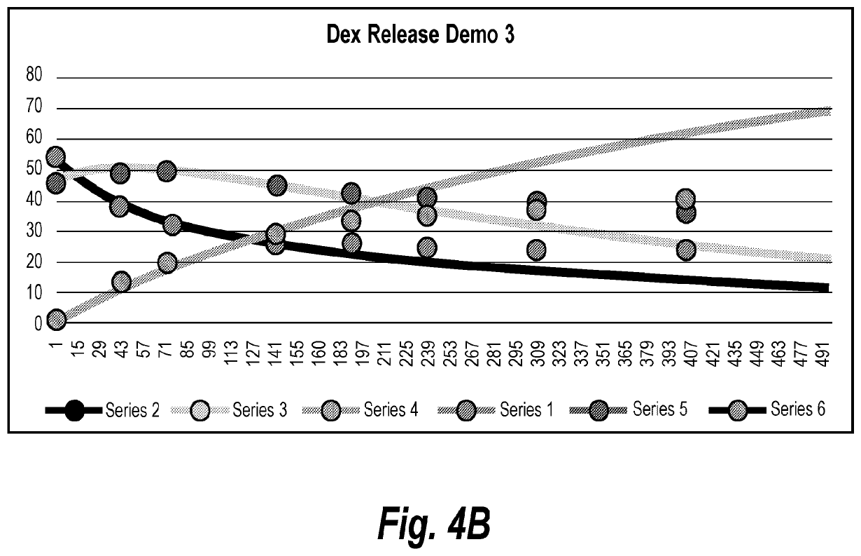

An empirical model was developed to fit the Dex release data at early time points (before the back reaction becomes significant). Two features of the P-Dex release system were built into the model: 1) Different release rates for the two diastereomers, and 2) equilibration of the two diastereomers without release. The second parameter was required to explain the increase in the thermodynamically preferred, slow-releasing diastereomer that occurred in the kinetics of Demo lot 3. The modeled (lines) and actual (points) kinetic data are shown in the charts below, in which the purple line is the Dex release rate, the green line is the slow-releasing diastereomer, and the red line is the fast-releasing diastereomer (FIGS. 4A and 4B).

The relative rates of release (at pH=4) are 3:1, and the release rate is comparable to the rate of equilibration of the two diastereomers. Because the slow-releasing diastereomer is thermodynamically favored, the greatest difference in release rate is seen in the first three days of the experiment. These results demonstrate that P-Dex polymers with varying initial release rates can be synthesized by manipulating the ratios of the diastereomeric monomers in the polymer.

### Example 7. Effect of Diastereomerically-enriched P-Dex Polymers in Mitigating Symptoms of Collagen-induced Rheumatoid Arthritis (CIA) in Rats

The rat collagen induced arthritis (CIA) model is induced by a primary injection followed by a boost immunization with collagen. The boost injection is given on Day 7 and erosive polyarthritis becomes evident starting Day 14 after the primary immunization. Rats are expected to show swelling and/or redness of the limbs as well as limited usage.

The objective of the study is to evaluate the therapeutic effect of diastereomerically enriched mixtures of PDEX compositions on arthritic rats.

### Experimental Procedure:

On arrival, animals were group housed (up to 3 animals of the same dosing group together) in polycarbonate cages containing appropriate bedding equipped with an automatic watering valve. On Day 7 following the boost injection, the animals were placed into new clean cages on soft bedding (Alphadri, SS1010, Shepherd Specialty). Primary enclosures were as described in the Guide for the Care and Use of Laboratory Animals. The temperature in the testing environment was kept at 19° C. to 25° C., with humidity being kept 30 to 70%, and the light cycle was kept at 12 hours light and 12 hours dark.

### Preparation of Collagen Emulsion:

The day before the emulsion is to be made 83.35 mL 0.05M acetic acid was added to 500 mg bovine type II collagen and placed on a stir plate in a cold room. It was critical to keep the collagen cold at all times as to prevent denaturation of the protein. The required volume of 6 mg/ml collagen solution was poured into a 50 ml conical tube; and the same volume of IFA was added to yield a final emulsion of 3 mg/ml. While conical tube was still on ice a Polytron (Kinematica AG, model PT 2100, or suitable alternative) ˜30,000 rpm was used to emulsify contents, making sure to move the tip around constantly to fully incorporate all material. It took roughly 5-6 minutes to achieve the proper emulsification. The temperature was kept below 4° C. during emulsification process. In order to avoid overheating of the Polytron probe, the following steps were used to homogenize:
homogenize 2 minutes and stop for 3-4 minutes, repeat the step again;
during homogenization verify that the probe is cold to touch by placing the hand on the upper part close to the motor;
if the probe is warm to touch before the end of the 2 minutes homogenization period, stop the Polytron and wait until it cools down.
   These steps were repeated making sure not to homogenize more than 6 min in total. To test for proper emulsification, 2-4 minutes were allowed to pass after the last homogenization cycle and then a drop of emulsified material was placed into ice cold water; if the material maintains a tight ball from which there is no oil sheen dispersion, then the emulsification was complete; otherwise the procedure was continued.

### Preparation of Vehicle/Test Item:

Formulations of diastereomerically-enriched PDEX preparations were prepared the day of use by dissolving the required amount in 1× PBS (adjusted to pH 8). Gentle swirling was used if required. The solution was filtered using a 0.22 μm PVDF filter and used within 8 hours of preparation.

Dexamethasone formulation was prepared by dissolution in 0.5% HPMC in UPW and used within 3 days.

### Primary Immunization:

Rats were anesthetized with isoflurane, and were slowly injected with 0.3 mL of the emulsion intradermally in the lower back (i.e. 900 μg collagen II) just proximal to the tail base and returned to their cages. Using a sterile 1 ml syringe and appropriately sized needle (25 or 27 gauge×⅝″), the syringe was filled with 0.7mL (enough for two animals) of the emulsion. The anesthesia regulator was set to 5% and the oxygen flow to 2.5 L/min. Once the anesthesia chamber was pre-charged with anesthetic, the regulator was reduced to ˜2.5%. Anesthesia was confirmed by pinching one of the hind paws and looking for pain reflex. The lower back area, which was shaved, was then cleaned with 70% ethanol followed by sterile water. The prepared area was then injected intradermally with a 25 or 27 g needle in 2 different sites approximately equidistant from midline, each injection containing 0.15 mL of material (900 μg collagen II per animal); the injections were on either side of the spine at the base of the tail.

### Boost Injection:

On Day 7, rats were anesthetized with isoflurane. The animals received boost injections with type II collagen (2 injections of 0.15 mL of material per site—900 μg collagen II per animal) emulsified in incomplete Freund's adjuvant (IFA) administered intradermally (lower back), proximal to the first immunization sites. Animals were monitored continually for recovery from anesthesia until animals could maintain sternal recumbency.

### Animal Randomization:

Animals will be randomized on Day 16, based on paw volume.

### Dose Administration:

Following stratification animals were dosed using the therapeutic regimen. The dosing was performed for up to 21 days starting on Day 17.

Groups 1 and 2 animals received the vehicle (1× PBS pH 8) once on Day 17 at a dose volume of 10 mL/kg.

Groups 3 to 5 received the test/reference items once on Day 17 at a dose volume of 6 mL/kg and Group 6 received test item once on Day 17 at a dose volume of 3 mL/kg. Dosings were performed intravenously by the tail vein.

Group 7 received dexamethasone solution daily for 5 days (Days 17 to 21 inclusively). Group 8 received dexamethasone solution daily for 21 days (Days 17 to 37 inclusively). All dosings were given by oral gavage at a dose volume of 10 ml/kg.

### Clinical Observations/Body Weights:

Upon arrival, morbidity/mortality checks were performed at least once daily (AM). A weekly detailed observation was also performed. Body weights were recorded at least once prior to dosing, on the day of primary immunization and weekly thereafter. Body weights were collected on the day of termination.

### Disease Scoring and Paw Volume:

Visual clinical scoring was performed on all limbs and for the hindlimbs paw volume measurement by plethymometer was performed. Scoring and volume measurement were performed on Days 1, 7, 11 and three times per week starting on Day 16 following the scoring protocol below:
Scoring 0 to 4
(0) Normal
(1) Erythema and mild swelling confined to the mid-foot (tarsals) or ankle joint, or digits.
(2) Erythema and mild swelling extending from the ankle to the mid-foot. (2 segments).
(3) Erythema and moderate swelling extending from the ankle to the metatarsal joints. (2 segments).
(4) Erythema and severe swelling encompassing the ankle, foot, and digits.

### Tissue Collection and Preservation:

All 4 legs were collected, weighed and retained in neutral buffered 10% formalin. One hindlimb was transferred in 70% alcohol 72 hours after termination for micro-CT scanning. In addition, L2-L6 vertebras were collected and placed in 70% alcohol for micro-CT scanning. One hindlimb and one vertebra (L5) from all rats were scanned using a high resolution Micro-CT system (SCANCO Medical) and analysed for bone volume and bone density.

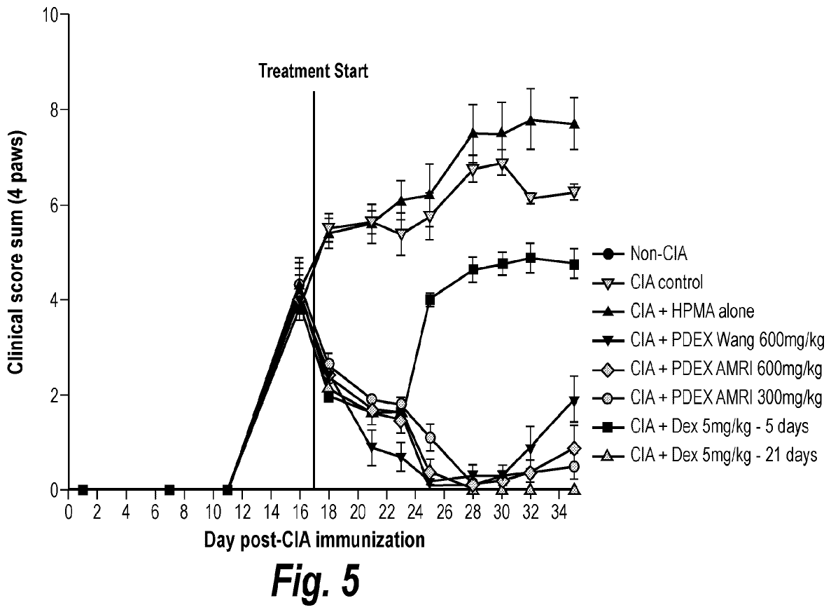

As was expected, the rats treated with diastereomerically enriched P-Dex polymers showed a statistically significant diminution of symptoms as compared to untreated rats, rats treated with vehicle, and rats treated with compositions of non-diastereomerically enriched P-Dex polymers (See FIG. 5).

### Example 8. Stability

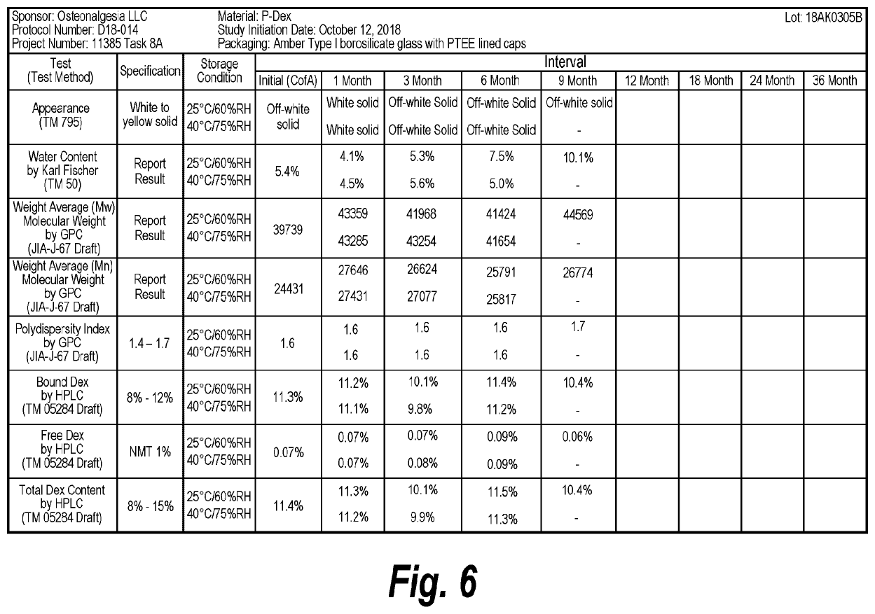

The P-Dex material was stores in an Amber-Type 1 borosilicate glass container with a PTFE lined cap. Initial tests were done to determine: appearance, water content, weight average molecular weight, number average molecular weight, polydispersity index, bound Dex, free Dex, and total Dex. Follow-up experiments were done for all these tests at the 1, 3, 6 and 9-month mark. After the 9-month mark, the material had shown very little water uptake, and good stability with respect to the bound vs. free Dex (See FIG. 6).

Additional embodiments are within the claims.

What is claimed is:

1. A composition comprising a mixture of diastereomers of compounds VIa and VIb, wherein the diastereomers are present in the mixture in a diastereomeric ratio (d.r.) of at least 60:40 Formula VIa:Formula VIb, or are present in a d.r. of at or at least 60:40 of Formula VIb:Formula VIa,
wherein the diastereomer of Formula (VIa) is: 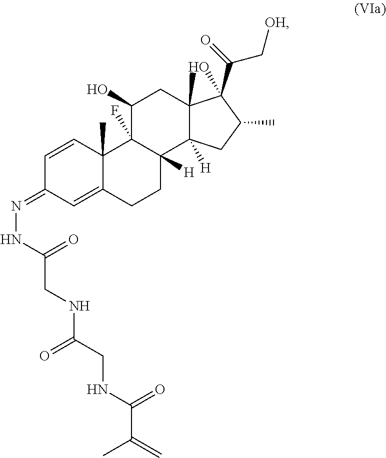 [SMILES: C=C(C)C(=O)NCC(=O)NCC(=O)N/N=C1/C=C[C@@]2(C)C(=C1)CC[C@H]1[C@@H]3C[C@@H](C)[C@](O)(C(=O)CO)[C@@]3(C)C[C@H](O)[C@@]12F]
and wherein the diastereomer of Formula (VIb) is: 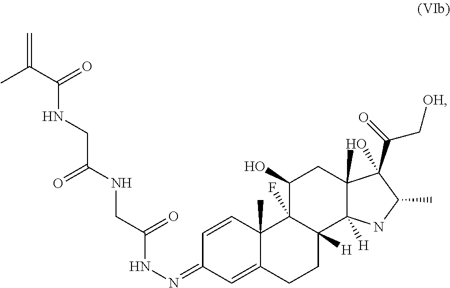 [SMILES: C=C(C)C(=O)NCC(=O)NCC(=O)N/N=C1\C=C[C@@]2(C)C(=C1)CC[C@H]1[C@@H]3N[C@@H](C)[C@](O)(C(=O)CO)[C@@]3(C)C[C@H](O)[C@@]12F]
or pharmaceutically acceptable salts thereof.

2. The composition of claim 1, wherein the diastereomers are present in the composition in a d.r. of at least 65:35 of Formula VIa:Formula VIb, or are present in a d.r. of at or at least 65:35 of Formula VIb:Formula VIa.

3. The composition of claim 1, wherein the diastereomers are present in the composition in a d.r. of at least 70:30 of Formula VIa:Formula VIb, or are present in a d.r. of at least 70:30 of Formula VIb:Formula VIa.

4. The composition of claim 1, wherein the diastereomers are present in the composition in a d.r. of at least 75:25 of Formula VIa:Formula VIb, or are present in a d.r. of at least 75:25 of Formula VIb:Formula VIa.

5. The composition of claim 1, wherein the diastereomers are present in the composition in a d.r. of at least 80:20 of Formula VIa:Formula VIb, or are present in a d.r. of at least 80:20 of Formula VIb:Formula VIa.

6. The composition of claim 1, wherein the diastereomers are present in the composition in a d.r. of at least 85:15 of Formula VIa:Formula VIb, or are present in a d.r. of at least 85:15 of Formula VIb:Formula VIa.

7. The composition of claim 1, wherein the diastereomers are present in the composition in a d.r. of at least 90:10 of Formula VIa:Formula VIb, or are present in a d.r. of at least 90:10 of Formula VIb:Formula VIa.

8. The composition of claim 1, wherein the diastereomers are present in the composition in a d.r. of at least 95:5 of Formula VIa:Formula VIb, or are present in a d.r. of at least 95:5 of Formula VIb:Formula VIa.

9. The composition of claim 1, wherein the diastereomers are present in the composition in a d.r. of at least 98:2 of Formula VIa:Formula VIb, or are present in a d.r. of at least 98:2 of Formula VIb:Formula VIa.

10. The composition of claim 1, wherein the diastereomers are present in the composition in a d.r. of at least 99:1 of Formula VIa:Formula VIb, or are present in a d.r. of at least 99:1 of Formula VIb:Formula VIa.

11. The composition of claim 1, wherein the diastereomers are present in the composition in a d.r. of at least 99.9:0.1 of Formula VIa:Formula VIb, or are present in a d.r. of at least 99.9:0.1 of Formula VIb:Formula VIa.

12. The composition of claim 1, wherein one diastereomer has a release profile corresponding to the early releasing diastereomer profile shown in FIG. 1.

13. The composition of claim 1, wherein one diastereomer has a release profile corresponding to the late releasing diastereomer profile shown in FIG. 1.

14. The composition of claim 1, wherein one diastereomeric form has a release profile matching the early releasing profile shown in FIG. 1.

15. A process for preparing an HPMA-dexamethasone copolymer conjugate, the process comprising:
(a) contacting in a solution a compound of Formula (IV) 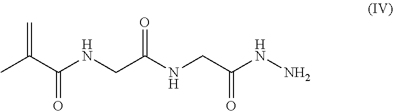
with a compound of Formula (V) 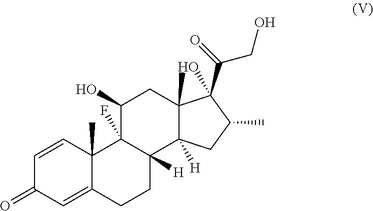 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)CO]
to form a compound of Formula (VI), 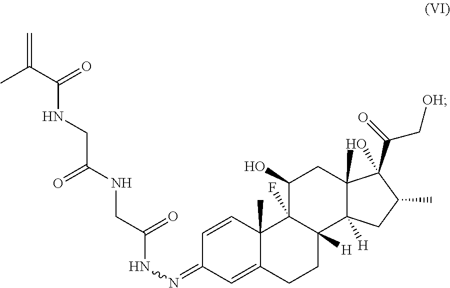 [SMILES: C=C(C)C(=O)NCC(=O)NCC(=O)NN=C1C=C[C@@]2(C)C(=C1)CC[C@H]1[C@@H]3C[C@@H](C)[C@](O)(C(=O)CO)[C@@]3(C)C[C@H](O)[C@@]12F]
wherein the compound of Formula (VI) comprises diastereomeric compounds of Formula (VIa) and Formula (VIb): 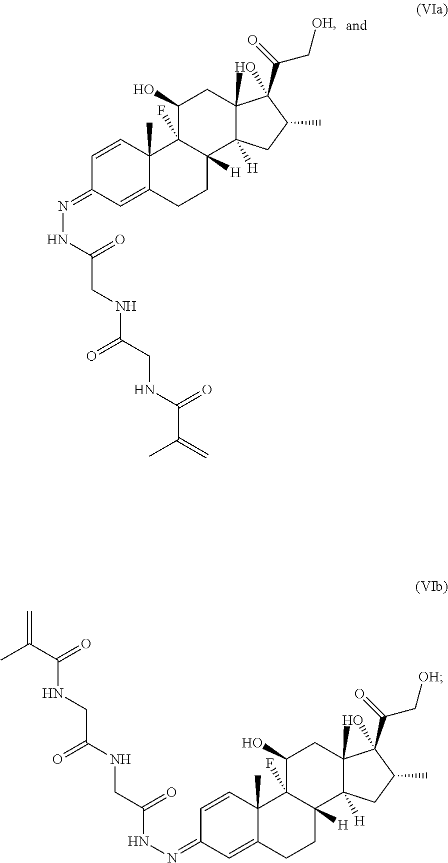
(b) removing from the solution the less soluble diastereomer to obtain a solution of VIa and VIb which is diastereomerically enriched with the more soluble diastereomer and a solid that is diastereomerically enriched in the less soluble diastereomer:
(c) optionally equilibrating the non-desired diastereomer, and repeating steps (b) and (c); and
(d) contacting the mixture of Formula (VIa) and (VIb) with a compound of Formula (VII) 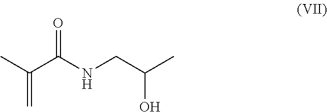
to form an HPMA-dexamethasone copolymer conjugate enriched in one diastereomer of the HPMA-dexamethasone copolymer conjugate.

16. A process for preparing a monomer of Formula VIa or Formula VIb, the process comprising:
(a) contacting in a solution a compound of Formula (IV) 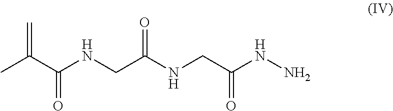
with a compound of Formula (V) 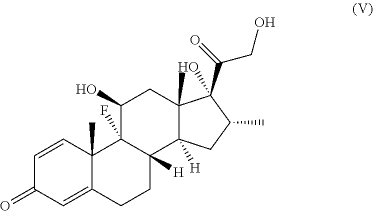 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)CO]
to form a compound of Formula (VI), 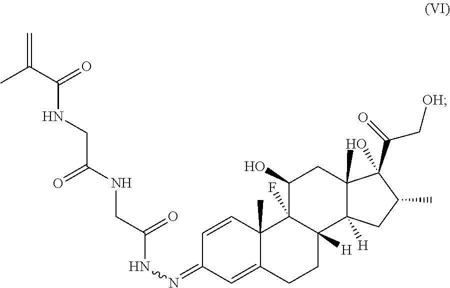 [SMILES: C=C(C)C(=O)NCC(=O)NCC(=O)NN=C1C=C[C@@]2(C)C(=C1)CC[C@H]1[C@@H]3C[C@@H](C)[C@](O)(C(=O)CO)[C@@]3(C)C[C@H](O)[C@@]12F]
wherein the compound of Formula (VI) comprises diastereomeric compounds of Formula (VIa) and Formula (VIb): 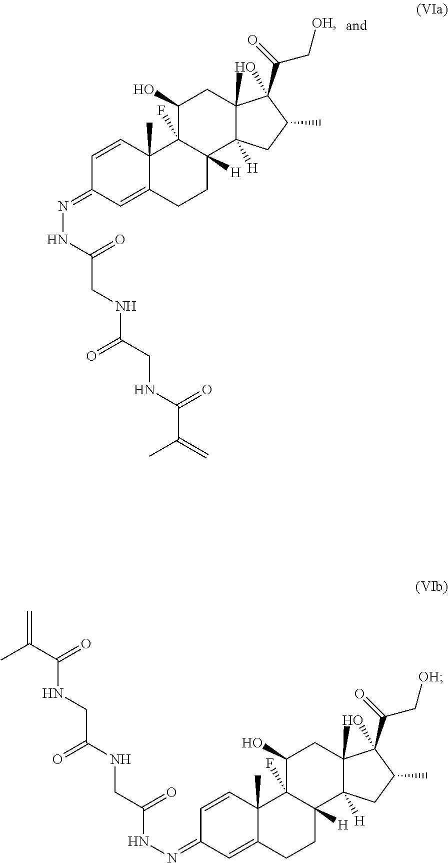
(b) removing from the solution the less soluble diastereomer to obtain a solution of VIa and VIb which is diastereomerically enriched in the more soluble diastereomer and a solid that is diastereomerically enriched in the less soluble diastereomer.

17. The process of claim 16, further comprising racemizing the less-soluble diastereomer, and repeating steps (a) and (b).

18. The process of claim 16, wherein the solution of step (a) comprises a polar protic solvent.

19. The process of claim 18, wherein the solvent is methanol.

20. The process of claim 16, wherein the solution of step (a) comprises a catalyst.

21. The process of claim 20, wherein the catalyst is an acid catalyst.

22. The process of claim 21, wherein the acid catalyst is hydrochloric acid.

23. The process of claim 16, wherein the less-soluble diastereomer of step (b) is removed from the solution as a solid.

24. The process of claim 16, wherein the less-soluble diastereomer of step (b) is recrystallized in THF and MTBE.

25. The process of claim 16, wherein the solid obtained in step (b) is a ratio of 60:1 less-soluble to more-soluble diastereomer.

26. The process of claim 16, wherein step (c) is performed by heating the compound of Formula (VIa) or the compound of Formula (VIb) in methanol to obtain a mixture of the diastereomers, in which each diastereomer makes up more than 40 mole percent of the diastereomers in the mixture.

27. The process of claim 16, wherein the solvent is heated to 65° C.

28. The process of claim 15, wherein step (d) is performed with a chain transfer agent and a free radical initiator.

29. The process of claim 28, wherein the chain transfer agent is CTA RAFT.

30. The process of claim 28, wherein the free radical initiator is AIBN.

31. A composition comprising a mixture of HPMA-dexamethasone conjugate diastereomers, wherein the composition is enriched for one diastereomer of an HPMA-dexamethasone conjugate.

32. The composition of claim 31, wherein the mixture comprises HPMA-dexamethasone conjugate diastereomers that differ in their dexamethasone release rates.

33. A composition of Formula (III) comprising a mixture of HPMA-dexamethasone conjugate copolymers, wherein the composition comprises interspersed diastereomers B and C or a pharmaceutically acceptable salt thereof,
wherein the polymer of Formula (III) is: 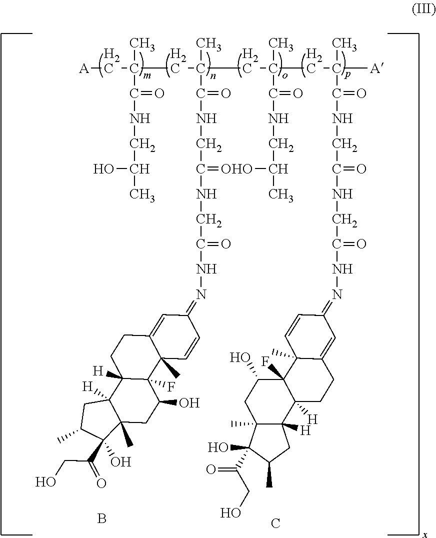
wherein:
m is an integer between 0 or 1 and 1000;
n is an integer between 0 or 1 and 1000;
o is an integer between 0 or 1 and 1000;
p is an integer between 0 or 1 and 1000;
X is an integer between 0 or 1and 1000;
wherein A and A′ are each, independently, selected from H or a termination product of a radical initiator, provided that at least one A or A′ is H; and
wherein the diastereomers comprising each of the copolymers are present in the mixture in a diastereomeric ratio (d.r.) of at least 60:40 B:C, or are present in a d.r. of at or at least 60:40 of C:B,
or a pharmaceutically acceptable salt thereof.

34. The composition of claim 33, wherein the diastereomers are present in the composition in a d.r. of at least 65:35 of B:C, or are present in a d.r. of at or at least 65:35 of C:B.

35. The composition of claim 33, wherein the diastereomers are present in the composition in a d.r. of at least 70:30 of B:C, or are present in a d.r. of at least 70:30 of C:B.

36. The composition of claim 33, wherein the diastereomers are present in the composition in a d.r. of at least 75:25 of B:C, or are present in a d.r. of at least 75:25 of C:B.

37. The composition of claim 33, wherein the diastereomers are present in the composition in a d.r. of at least 80:20 of B:C, or are present in a d.r. of at least 80:20 of C:B.

38. The composition of claim 33, wherein the diastereomers are present in the composition in a d.r. of at least 85:15 of B:C, or are present in a d.r. of at least 85:15 of C:B.

39. The composition of claim 33, wherein the diastereomers are present in the composition in a d.r. of at least 90:10 of B:C, or are present in a d.r. of at least 90:10 of C:B.

40. The composition of claim 33, wherein the diastereomers are present in the composition in a d.r. of at least 95:5 of B:C, or are present in a d.r. of at least 95:5 of C:B.

41. The composition of claim 33, wherein the diastereomers are present in the composition in a d.r. of at least 98:2 of B:C, or are present in a d.r. of at least 98:2 of C:B.

42. The composition of claim 33, wherein the diastereomers are present in the composition in a d.r. of at least 99:1 of B:C, or are present in a d.r. of at least 99:1 of C:B.

43. The composition of claim 33, wherein the diastereomers are present in the composition in a d.r. of at least 99.9:0.1 of B:C, or are present in a d.r. of at least 99.9:0.1 of C:B.

44. The composition of claim 33, wherein the overall release rate of dexamethasone from the composition at hour 24 is greater than 25% of a racemic mixture of HPMA-dexamethasone conjugate copolymers.

45. The composition of claim 33, wherein the overall release rate of dexamethasone from the composition at hour 24 is less than 25% of a racemic mixture of HPMA-dexamethasone conjugate copolymers.

46. A pharmaceutical composition comprising the composition of claim 33 and a pharmaceutically acceptable carrier.

47. The pharmaceutical composition of claim 46 formulated for delivery to a human subject.

48. A method of treating an inflammatory disease in a subject in need thereof comprising administering to the subject a therapeutically effective amount of an HPMA-dexamethasone copolymer conjugate enriched in one diastereomer of the HPMA-dexamethasone copolymer conjugate.

49. The method of claim 49, wherein the inflammatory disease is rheumatoid arthritis.

50. A method of treating an inflammatory disease in a subject in need thereof comprising administering to the subject a therapeutically effective amount the mixture of HPMA-dexamethasone conjugate copolymers of any one of claims 33-45 or the pharmaceutical composition of claim 46.

51. The method of claim 50, wherein the inflammatory disease is rheumatoid arthritis.
